(19) United States
(12) Patent Application Publication
(10) Pub. No.: US 2025/0134849 A1
(43) Pub. Date: May 1, 2025

(54) PHARMACEUTICAL COMPOSITION FOR PREVENTING OR TREATING OCULAR DISEASE COMPRISING ENAVOGLIFLOZIN

(71) Applicant: DAEWOONG THERAPEUTICS INC., Hwaseong-si (KR)

(72) Inventors: Sanghan PARK, Seoul (KR); Bokki KANG, Seongnam-si (KR); Donghwan KIM, Suwon-si (KR); Minhyung KANG, Yongin-si (KR)

(73) Assignee: DAEWOONG THERAPEUTICS INC., Hwaseong-si (KR)

(21) Appl. No.: 18/496,199

(22) Filed: Oct. 27, 2023

(51) Int. Cl. A61K 31/351; A61K 9/00; A61K 47/10; A61K 47/22; A61K 47/26; A61P 27/02

(52) U.S. Cl. CPC A61K 31/351; A61K 9/0048; A61K 47/10; A61K 47/22; A61K 47/26; A61P 27/02

(57) ABSTRACT

The present invention provides a method for treating macular degeneration which comprises administering enavogliflozin or a pharmaceutically acceptable salt thereof; and a pharmaceutical composition for treating macular degeneration comprising enavogliflozin or a pharmaceutically acceptable salt thereof as an active ingredient.

### TECHNICAL FIELD

The present invention relates to a method for treating macular degeneration which comprises administering enavogliflozin or a pharmaceutically acceptable salt thereof; and a pharmaceutical composition for treating macular degeneration comprising enavogliflozin or a pharmaceutically acceptable salt thereof as an active ingredient.

### BACKGROUND ART

The eye is composed of various complex tissues. It is known in the art that there are limitations in delivering a pharmacologically active ingredient into the ocular tissues simply by instilling the active ingredient, due to various factors that interfere with drug delivery thereto. Therefore, ocular diseases treated or ameliorated with an eye drop formulation are very limited. Currently, commercially available pharmaceutical products in the form of eye drop formulation are used mainly for treating or ameliorating anterior segment eye diseases, such as corneal conjunctival diseases.

Treating or ameliorating posterior segment eye diseases (PSEDs), such as diabetic eye diseases, macular degeneration, etc., requires invasive repeated injections to the eye. For example, antibody-containing pharmaceutical products are administered through repeated intravitreal injections, for treating or ameliorating PSEDs. However, the direct injections to the eye have many drawbacks, such as limited dose, very low patients' compliance due to invasive repeated injections, high risks of bleeding, pain, infection, and retinal detachment, high burden of therapeutic cost, and so on. Therefore, many patients fail to receive the repeated intraocular/intravitreal injections, which eventually leads to gradual blindness.

Macular degeneration occurs mainly in people over 50 years of age and is on the rise in the world. Although the causes of macular degeneration are not yet found accurately, it is known in the art that the risk factors thereof include age, smoking, hypertension, obesity, genetic predisposition, excessive UV exposure, low blood antioxidant concentration, and the like. It has been reported that the intravitreal injection of an anti-VEGF antibody or a peptide improves patients' vision and prognosis in the therapy of macular degeneration. However, said intravitreal injection also causes the inconvenience of having to undergo repeated injections every month, due to the short half-life thereof. And, it has been reported that the surgical method involving direct administration into the vitreous cavity may bring about complications such as cataracts, endophthalmitis, vitreous hemorrhage, retinal detachment, and so on.

Therefore, there is a need to develop a therapeutic agent and/or a formulation capable of addressing the side effects during the treatment of eye diseases such as macular degeneration and improving patients' compliance.

### DISCLOSURE

### Technical Problem

It is an object of the present invention to provide a method for treating macular degeneration comprising administering enavogliflozin or a pharmaceutically acceptable salt thereof to a mammal.

It is another object of the present invention to provide a pharmaceutical composition for treating macular degeneration comprising enavogliflozin or a pharmaceutically acceptable salt thereof as an active ingredient.

### Technical Solution

According to an aspect of the present invention, there is provided a method for treating macular degeneration in a mammal, which comprises administering a therapeutically effective amount of enavogliflozin or a pharmaceutically acceptable salt thereof to the mammal in need thereof.

According to another aspect of the present invention, there is provided a pharmaceutical composition comprising a therapeutically effective amount of enavogliflozin or a pharmaceutically acceptable salt thereof and a pharmaceutically acceptable excipient, wherein the pharmaceutical composition is for treating macular degeneration.

### Advantageous Effects

It has been found by the present invention that enavogliflozin or a pharmaceutically acceptable salt thereof can be delivered to the vitreous body, retina, and choroid and maintain in a high concentration therein when administered (or instilled) in the form of eye drop formulation, thereby exhibiting excellent therapeutic effects on macular degeneration. And, it has been found by the present invention that an eye drop formulation comprising enavogliflozin or a pharmaceutically acceptable salt thereof even in a low amount shows excellent therapeutic effects against macular degeneration through the effective exposure to the retina and choroid, along with extremely limited systemic exposure. In addition, unlike conventional pharmaceutical products requiring repeated intravitreal injections, the composition of the present invention may be conveniently administered to the eye in the form of eye drop formulation, thereby improving patients' compliance.

### DESCRIPTION OF DRAWINGS

FIG. 1 shows the results obtained by measuring the concentrations of the active ingredient in the samples (plasma, vitreous body, and retina+choroid) of each test group at 1, 4, 8, 16, and 24 hours after administration of the compositions of the present invention.

FIG. 2 shows the results obtained by measuring the fluorescence intensities at day 7 after administration of the compositions of the present invention according to Experimental Example 2.

FIG. 3 shows the fundus photographs taken at day 7 after administration of the compositions of the present invention according to Experimental Example 2.

FIG. 4 shows the results obtained by measuring the fluorescence intensities at day 14 after administration of the compositions of the present invention according to Experimental Example 2.

FIG. 5 shows the fundus photographs taken at day 14 after administration of the compositions of the present invention according to Experimental Example 2.

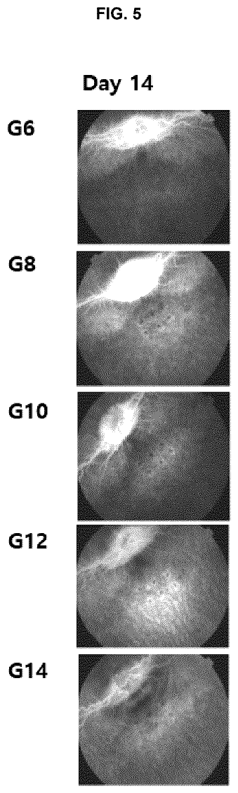

### BEST MODE FOR CARRYING OUT THE INVENTION

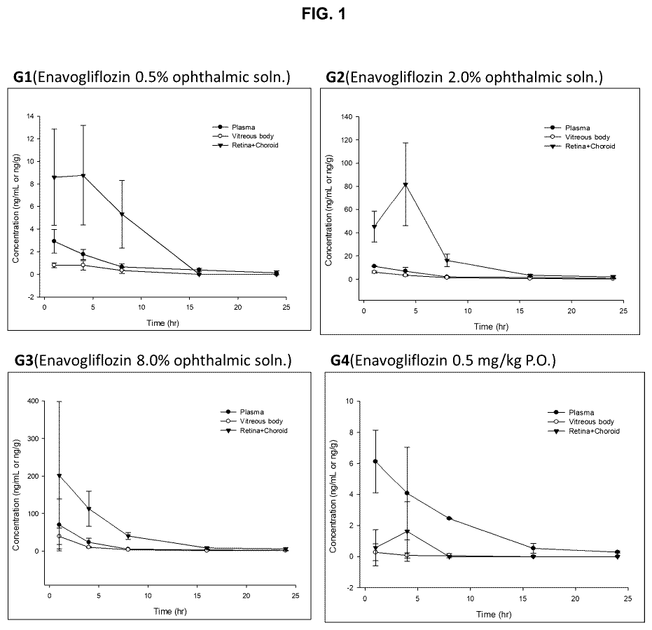

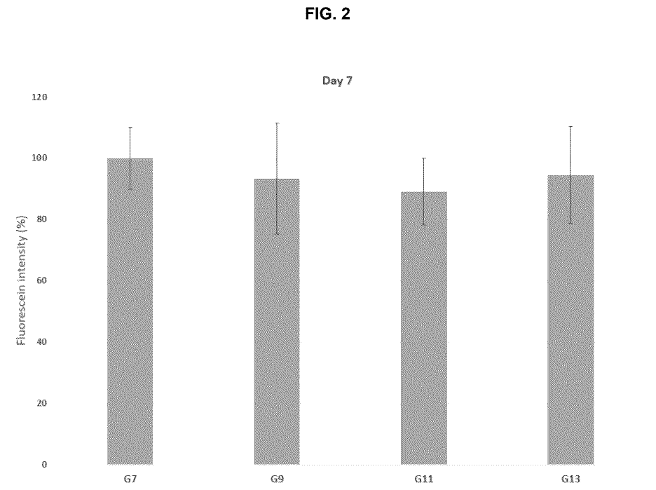

It has been found by the present invention that the administration (i.e., instillation) of enavogliflozin-containing eye drop formulations can be delivered to the vitreous body, retina, and choroid with high permeability thereto, which leads to a high concentration of the active ingredient therein (FIG. 1). It has been also found by the present invention that the eye drop formulations according to the present invention show a rapid and extended therapeutic and/or ameliorating effect from the beginning of administration (FIG. 2 to 4). Therefore, enavogliflozin or a pharmaceutically acceptable salt thereof and a pharmaceutical composition (such as an eye drop formulation) comprising the same may be usefully applied for treating or ameliorating macular degeneration.

In the present invention, enavogliflozin has the chemical structure of Formula 1 below.

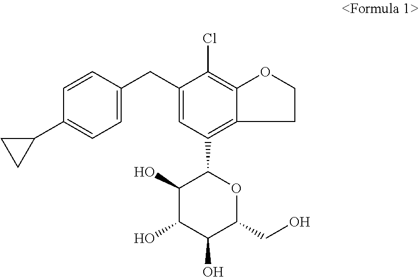 [SMILES: OC[C@H]1O[C@@H](c2cc(Cc3ccc(C4CC4)cc3)c(Cl)c3c2CCO3)[C@H](O)[C@@H](O)[C@@H]1O]

The macular degeneration is a medical condition which may result in blurred or no vision in the center of the visual field. The degeneration includes pathological or anatomical deterioration and/or modification as well as the processes of replacing existing normal tissues with tissues having different natures. The macular is concentrated on the center of the retina to detect light and color and the macular degeneration includes a situation in which vision is reduced by being replaced with the tissues which cannot detect light and color (e.g., scar). The macular degeneration includes age-related macular degeneration (AMD), macular dystrophy due to genetic causes, myopic macular degeneration due to high myopia, and macular degeneration occurred by inflammatory diseases and trauma, but not limited thereto. The age-related macular degeneration is a degeneration of the macular caused by the aging of the retina. The age-related macular degeneration includes both dry (non-exudative) macular degeneration and wet (exudative) macular degeneration. Dry (non-exudative) age-related macular degeneration involves drusen, cellular debris in their macula that gradually damages light-sensitive cells and leads to vision loss. In wet (exudative) macular degeneration, blood vessels grow under the macula, causing blood and fluid to leak into the retina. As used herein, the expression ‘macular degeneration’ includes any and all forms of macular degeneration mentioned in the above.

The present invention provides a method for treating macular degeneration in a mammal, which comprises administering a therapeutically effective amount of enavogliflozin or a pharmaceutically acceptable salt thereof to the mammal in need thereof.

In the method according to the present invention, the active ingredient, i.e., enavogliflozin or a pharmaceutically acceptable salt thereof, may be delivered to a vitreous body, a retina, and/or a choroid when administered to the mammal in need thereof.

In the method according to the present invention, the active ingredient, i.e., enavogliflozin or a pharmaceutically acceptable salt thereof, may be administered in an eye drop formulation. That is, the administering may be carried out by administering an eye drop formulation comprising a therapeutically effective amount of enavogliflozin or a pharmaceutically acceptable salt thereof to the mammal in need thereof. The eye drop formulation may be in a solution form or in a suspension form. In an embodiment, the eye drop formulation may be in a solution form. In another embodiment, the eye drop formulation may further comprise one or more excipients selected from the group consisting of polyoxyl castor oil such as polyoxyl 35 castor oil (Kolliphor® ELP), polysorbate such as polysorbate 80 (Tween® 80), D-α-tocopheryl polyethylene glycol succinate, and glycerin.

In the method according to the present invention, the active ingredient, i.e., enavogliflozin or a pharmaceutically acceptable salt thereof, may be administered in the form of eye drop formulation having a concentration of about 0.25 to 10 wt/v %, preferably about 1.5 to 8 wt/v %, although the concentration may be changed according to the patient's age, sex, health condition, severity of disease, etc. The administration (e.g., instillation) can be carried out in an appropriate interval, e.g., in a single dose or in divided doses per day, according to the doctor's or pharmacist's instruction.

The present invention includes, within its scope, a pharmaceutical composition comprising a therapeutically effective amount of enavogliflozin or a pharmaceutically acceptable salt thereof and a pharmaceutically acceptable excipient, wherein the pharmaceutical composition is for treating macular degeneration.

In the pharmaceutical composition according to the present invention, the active ingredient, i.e., enavogliflozin or a pharmaceutically acceptable salt thereof, may be delivered to a vitreous body, a retina, and/or a choroid when administered to the mammal in need thereof.

In a preferred embodiment, the pharmaceutical composition according to the present invention may be in the form of eye drop formulation. The eye drop formulation may be in a solution form or in a suspension form. In an embodiment, the eye drop formulation may be in a solution form. In another embodiment, the eye drop formulation may further comprise a pharmaceutically acceptable excipient, such as a solubilizer, a stabilizer, a buffering agent, a pH controlling agent. For example, the pharmaceutically acceptable excipient may comprise one or more excipients selected from the group consisting of polyoxyl castor oil such as polyoxyl 35 castor oil (Kolliphor® ELP), polysorbate such as polysorbate 80 (Tween® 80), D-α-tocopheryl polyethylene glycol succinate, and glycerin. If necessary, the pharmaceutical composition in the form of eye drop formulation may further comprise a buffering agent such as boric acid etc.; a pH controlling agent such as hydrochloric acid, sodium hydroxide, etc; a preservative, and so on. And, the pharmaceutical composition in the form of eye drop formulation may be sterilized according to conventional methods.

In the pharmaceutical composition according to the present invention, the active ingredient, i.e., enavogliflozin or a pharmaceutically acceptable salt thereof, may be administered in the form of eye drop formulation having a concentration of about 0.25 to 10 wt/v %, preferably about 1.5 to 8 wt/v %, although the concentration may be changed according to the patient's age, sex, health condition, severity of disease, etc. The administration (e.g., instillation) can be carried out in an appropriate interval, e.g., in a single dose or in divided doses per day, according to the doctor's or pharmacist's instruction.

Hereinafter, the present invention will be described in more detail through Examples and Experimental Examples. However, these Examples and Experimental Examples are provided for illustration purposes only, and are not intended to limit the scope of the invention.

### Examples: Preparation of Eye Drop Formulations Comprising a SGLT-2 Inhibitor

The excipients and water for injection (about 4 mL) were weighed according to the following tables and added to a transparent glass vial. The resulting each mixture was heated to about 60 to 80° C. under stirring to prepare a transparent solution.

The active ingredient in each amount according to the following tables was weighed and slowly added to the respective glass vial to completely dissolve the active ingredient. Water for injection was added to the resulting solutions to make the total volume of each solution to 5 mL. The respective solutions were sterilized by filtering through a 0.2 μm PVDF (polyvinylidene fluoride) filter and then filled into a sterile container. Enavogliflozin, one of the SGLT-2 inhibitors, was used as the active ingredient. The resulting eye drop formulations are summarized in the following tables.

Example 1. Eye drop formulation containing 0.5% enavogliflozin

| | Amount (mg) |
|---|---|
| Enavogliflozin | 25 |
| Kolliphor ELP ® (Polyoxyl 35 castor oil) | 200 |
| Tween ® 80 | 100 |
| Vitamin E TPGS | 25 |
| Glycerin | 125 |
| Water for injection | Up to 5 mL |

Example 2. Eye drop formulation containing 2.0% enavogliflozin

| | Amount (mg) |
|---|---|
| Enavogliflozin | 100 |
| Kolliphor ® ELP (Polyoxyl 35 castor oil) | 200 |
| Tween ® 80 | 100 |
| Vitamin E TPGS | 25 |
| Glycerin | 125 |
| Water for injection | Up to 5 mL |

Example 3. Eye drop formulation containing 5.0% enavogliflozin

| | Amount (mg) |
|---|---|
| Enavogliflozin | 250 |
| Kolliphor ® ELP (Polyoxyl 35 castor oil) | 200 |
| Tween ® 80 | 100 |
| Vitamin E TPGS | 25 |
| Glycerin | 125 |
| Water for injection | Up to 5 mL |

Example 4. Eye drop formulation containing 8.0% enavogliflozin

| | Amount (mg) |
|---|---|
| Enavogliflozin | 400 |
| Kolliphor ® ELP (Polyoxyl 35 castor oil) | 200 |
| Tween ® 80 | 100 |
| Vitamin E TPGS | 25 |
| Glycerin | 125 |
| Water for injection | Up to 5 mL |

### Comparative Example 1: Enavogliflozin-Containing Oral Formulation

Enavogliflozin (2 mg) was dispersed in 0.5% (w/v) methyl cellulose solution (10 mL) to prepare an enavogliflozin-containing oral formulation, which was used for oral administration in the following Experimental Examples.

### Comparative Example 2: Aflibercept

Aflibercept (EYLEA®) was purchased and used as a positive control.

### Experimental Example 1: Pharmacokinetic Analyses

### 1-1. Experimental Animals

General symptoms for the purchased New Zealand White rabbits (male) were observed during the 8-day acclimation period, for confirming the health thereof. The 40 healthy rabbits were used for the tests. During the test period, the animals were raised at a temperature of 23±3° C., relative humidity of 55±15%, ventilation frequency of 10 to 20 times/hr, lighting time of 12 hours, and illuminance of 150 to 300 Lux. The environmental conditions such as temperature/humidity of the animal room, ventilation frequency and illuminance were measured regularly during the raising period. The body weights of rabbits were measured. Based on the ranked body weights, the rabbits were randomly divided so that the average body weights of each test group were distributed as evenly as possible.

### 1-2. Test Groups and Administrations

The administrations of the test groups are carried out as shown in Table 1 below. G1 to G3 are the groups in which the eye drop formulations of Examples 1, 2, and 4 were administered, respectively. G4 is the group in which the enavogliflozin-containing oral formulation of Comparative Example 1 was administered.

TABLE 1

| | | Number | | | | | |
|---|---|---|---|---|---|---|---|
| | | of | Administered | | | | |
| Group | Sex | Animals | material | Dose | | Volume | |
| G1 | Male | 10 | Eye drop | 1 | drop/eye | 50 | μL/eye |
| | | | formulation | | | | |
| | | | of Example 1 | | | | |
| G2 | Male | 10 | Eye drop | 1 | drop/eye | 50 | μL/eye |
| | | | formulation | | | | |
| | | | of Example 2 | | | | |
| G3 | Male | 10 | Eye drop | 1 | drop/eye | 50 | μL/eye |
| | | | formulation | | | | |
| | | | of Example 4 | | | | |
| G4 | Male | 10 | Oral | 0.5 | mg/kg | 2.5 | ml/kg |
| | | | formulation | | | | |

of Comparative
Example 1

The administration of each eye drop formulation was carried out, by single administration, on both eyes in an amount of 50 μL/eye. Each eye drop formulation was instilled into the center of the corneas of both eyes using a pipette. The oral administration was carried out in a single dose (2.5 mL/kg) by inserting an oral catheter into the stomach.

### 1-3. Blood Collection

Blood collection was performed through the jugular vein and approximately 1 mL of whole blood was collected at 1, 4, 8, 16, and 24 hours after the respective administrations. All processes from the blood collection to the plasma storage were carried out in an ice-cold condition at approximately 4° C. The blood samples were placed in a tube to which 10 μL of heparin (sodium heparin 500 IU/mL) was added and then centrifuged at 3,000 rpm at 4° C. for 10 minutes. The resulting supernatant (i.e., plasma) was separated and then dispensed into a microtube.

### 1-4. Sample Collection

After the blood collections at 1, 4, 8, 16, and 24 hours (at 5 points) after the respective administrations, the animals were euthanized and the eyes (i.e., the eyeballs) were isolated. The retina (including choroid) and the vitreous body of both eyes were respectively isolated and then placed in microtubes. Each sample was weighed and stored in a deep freezer set to approximately −70° C. or lower.

The concentrations of enavogliflozin over time in the plasma, vitreous, retina, and choroid of each group were determined according to a LC-MS/MS analysis. The pharmacokinetic parameters were calculated according to a non-compartmental analysis using the WinNonlin 4.2 (Pharsight Corp., Cary, NC, USA) software.

### 1-5. Results of the Pharmacokinetic Analyses

As shown in Table 2 and FIG. 1, the maximum plasma concentration (Cₘₐₓ) in the plasma, vitreous body, retina and choroid increased proportionally as the content of enavogliflozin increased. In particular, the groups administered with the eye drop formulations (including the G1 group administered with the eye drop formulation containing enavogliflozin in the lowest amount) showed higher levels of Cₘₐₓ value in the vitreous body, retina, and choroid, compared to the G4 group (the oral administration group). It was also confirmed that the concentrations in the vitreous body, retina, and choroid were maintained higher in the groups administered with the eye drop formulations (FIG. 1). These results show that the therapeutic effects against eye diseases can be effectively achieved through administering the eye drop formulations.

TABLE 2

| | | | Matrix | | |
|---|---|---|---|---|---|
| | Pharmacokinetic | | | Vitreous | Retina + |
| Group | parameters | Unit | Plasma | body | Choroid |
| G1 | Cₘₐₓ | (ng/mL or ng/g) | 2.91 | 0.79 | 8.77 |
| (Enavogliflozin | AUCₗₐₛₜ | (hr*ng/mL or hr*ng/g) | 19.52 | 5.04 | 58.51 |
| 0.5%) | AUC_{inf} | (hr*ng/mL or hr*ng/g) | 20.65 | NR | NR |
| | Tₘₐₓ | (hr) | 1 | 1 | 4 |
| | t_{1/2} | (hr) | 5.9 | NR | NR |
| | CL/F | (mg/head)/(hr*ng/mL or | 0.048 | NR | NR |
| | | hr*ng/mL) | | | |
| | AUCₑₓₜᵣₐₚ | (%) | 5.4 | NR | NR |
| G2 | Cₘₐₓ | (ng/mL or ng/g) | 11.15 | 6.19 | 81.84 |
| (Enavogliflozin | AUCₗₐₛₜ | (hr*ng/mL or hr*ng/g) | 74.49 | 39.70 | 509.25 |
| 2.0%) | AUC_{inf} | (hr*ng/mL or hr*ng/g) | 86.90 | 44.68 | 524.62 |
| | Tₘₐₓ | (hr) | 1 | 1 | 4 |
| | t_{1/2} | (hr) | 11.2 | 8.7 | 5.3 |
| | CL/F | (mg/head)/(hr*ng/mL or | 0.012 | 0.022 | 0.002 |
| | | hr*ng/mL) | | | |
| | AUCₑₓₜᵣₐₚ | (%) | 14.3 | 11.1 | 2.9 |
| G3 | Cₘₐₓ | (ng/mL or ng/g) | 69.56 | 39.29 | 201.86 |
| (Enavogliflozin | AUCₗₐₛₜ | (hr*ng/mL or hr*ng/g) | 285.40 | 150.26 | 1129.92 |
| 8.0%) | AUC_{inf} | (hr*ng/mL or hr*ng/g) | 311.45 | 161.75 | 1169.20 |
| | Tₘₐₓ | (hr) | 1 | 1 | 1 |
| | T_{1/2} | (hr) | 10.3 | 6.5 | 4.7 |
| | CL/F | (mg/head)/(hr*ng/mL or | 0.003 | 0.006 | 0.001 |
| | | hr*ng/mL) | | | |
| | AUCₑₓₜᵣₐₚ | (%) | 8.4 | 7.1 | 3.4 |
| G4 | Cₘₐₓ | (ng/mL or ng/g) | 6.12 | 0.27 | 1.62 |
| (Enavogliflozin | AUCₗₐₛₜ | (hr*ng/mL or hr*ng/g) | 46.54 | 0.95 | 3.59 |
| 0.5 mg/kg) | AUC_{inf} | (hr*ng/mL or hr*ng/g) | 48.57 | NR | NR |
| | Tₘₐₓ | (hr) | 1 | 1 | 4 |
| | t_{1/2} | (hr) | 5.0 | NR | NR |
| | CL/F | (mg/head)/(hr*ng/mL or | 10294 | NR | NR |
| | | hr*ng/mL) | | | |
| | AUCₑₓₜᵣₐₚ | (%) | 4.2 | NR | NR |

### Experimental Example 2: Pharmacological Effect of Enavogliflozin on Macular Degeneration

### 2-1. Preparation of Macular Degeneration Animal Model

Chinchilla rabbits (n=24, male) were raised at a temperature of 23±3° C., relative humidity of 55±15%, ventilation frequency of 10 to 20 times/hr, lighting time of 12 hours, and illuminance of 150 to 300 Lux. The environmental conditions such as temperature/humidity of the animal room, ventilation frequency and illuminance were measured regularly during the raising period. During the acclimation, administration, and observation periods, the rabbits were raised in a stainless-steel breeding box (W500×L800×H500 mm) at 1 animal per breeding box. The breeding boxes were cleaned every day and water bottles were exchanged every day.

A mydriatic agent (Mydriacyl® Eye Drops 1%) was instilled into the right eye of the rabbits, followed by anesthetizing. A laser (LIGHTLas 532, LIGHTMED, USA) was irradiated to the eye under the conditions of 532 nm and power of 150 mW for 0.1 second, to create the 6 spots centered on the optic nerve at approximately 6 o'clock.

### 2-2. Test Groups and Administrations

The administrations of the test groups are carried out as shown in Table 3 below. G5 and G6 are the normal control grouped that did not cause macular degeneration. G7 to G14 is the groups in which macular degeneration was induced. G5 and G6 were administered with a saline. G7 and G8 were administered with a vehicle [i.e., a solution containing all ingredients, except for the active ingredient (enavogliflozin)]. G9 and G10 were administered with the Comparative Example 2 (i.e., a positive control). G11 and G12 were administered with the eye drop formulation of Example 3. And, G13 and G14 were administered with the eye drop formulation of Example 4.

TABLE 3

| | | Number | | | | | | |
|---|---|---|---|---|---|---|---|---|
| | | of | Administered | Macular | Rute of | | | Observation |
| Group | Sex | Animals | material | degeneration | administration | Dose | | (day) |
| G5 | Male | 5 | Saline | N | — | — | | 7 |
| G6 | Male | 5 | Saline | N | — | — | | 14 |
| G7 | Male | 5 | Vehicle | Y | Instillation | — | | 7 |
| G8 | Male | 5 | Vehicle | Y | Instillation | — | | 14 |
| G9 | Male | 5 | Comparative | Y | Intravitreal | 2000 | μg | 7 |
| | | | Example 2 | | injection | | | |
| G10 | Male | 5 | Comparative | Y | Intravitreal | 2000 | μg | 14 |
| | | | Example 2 | | injection | | | |
| G11 | Male | 5 | Eye drop | Y | Instillation | 50 | μL/eye | 7 |
| | | | formulation of | | | | | |
| | | | Example 3 | | | | | |
| G12 | Male | 5 | Eeye drop | Y | Instillation | 50 | μL/eye | 14 |
| | | | formulation of | | | | | |
| | | | Example 3 | | | | | |
| G13 | Male | 5 | Eye drop | Y | Instillation | 50 | μL/eye | 7 |
| | | | formulation of | | | | | |
| | | | Example 4 | | | | | |
| G14 | Male | 5 | Eye drop | Y | Instillation | 50 | μL/eye | 14 |
| | | | formulation of | | | | | |
| | | | Example 4 | | | | | |

### 2-3. Fundus Fluorescein Angiography

On the 7th and 14th days after the start of administration in each group, a mydriatic agent (Mydriacyl® Eye Drops 1%) was instilled into the right eye of the rabbits, followed by anesthetizing. Approximately 1 mL of 2% fluorescein sodium salt solution was injected through the ear vein, and the images were taken within approximately 2 minutes using a fundus camera (TRC-50IX, TOPCON, Japan). The efficacies were evaluated through retinal fundus fluorescein angiography. It was confirmed that the fluorescence intensity levels on days 7 and 14 in all macular degeneration-induced groups were higher than those in the normal control groups, G5 and G6 (p<0.001 or p<0.05).

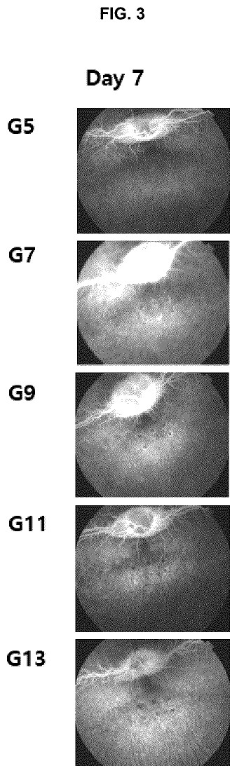

The average fluorescence intensities of the vehicle-administered groups (G7 and G8) were set to 100% and the relative levels (%) of the fluorescence intensities measured in each animal were analyzed. As shown in Table 4 below and FIGS. 2 and 3, on the 7th day of administration, the fluorescence intensity levels of G11 (the group administered with the eye drop formulation of Example 3) and G13 (the group administered with the eye drop formulation of Example 4) were significantly lower than that of G7 (the group administered with the vehicle). In particular, it can be confirmed that the fluorescence intensity of G11 was lower than that of G9 (the positive control group administered with the Comparative Example 2). These results show that, when the eye drop formulations of the present invention are instilled, significant therapeutic and/or ameliorating effects can be achieved in a faster time, compared to the intravitreal injection of Comparative Example 2.

TABLE 4
Fluorescence intensity at Day 7 (%)

| Group | G7 | G9 | G11 | G13 |
|---|---|---|---|---|
| Mean | 100.000 | 93.345 | 89.222 | 94.419 |
| Standard deviation | 10.182 | 18.079 | 10.955 | 15.805 |

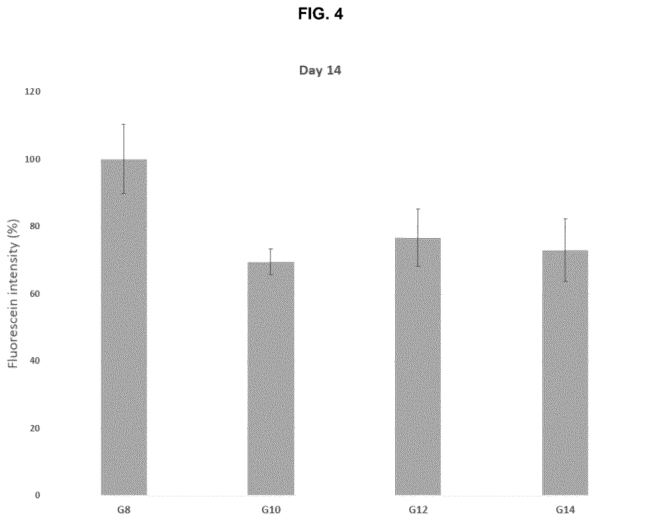

In addition, as shown in Table 5 below and FIGS. 3 and 4, on the 14th day of administration, the fluorescence intensity levels of G12 (the group administered with the eye drop formulation of Example 3) and G14 (the group administered with the eye drop formulation of Example 4) were also significantly lower than that of G8 (the group administered with the vehicle). And, it was confirmed that G12 and G14 showed a therapeutic effect similar to that of the positive control group, while maintaining the therapeutic effects that appeared quickly on the 7th day.

TABLE 5
Fluorescence intensity at Day 14 (%)

| Group | G8 | G10 | G12 | G14 |
|---|---|---|---|---|
| Mean | 100.000 | 69.403 | 76.604 | 72.833 |
| Standard deviation | 10.349 | 3.864 | 8.630 | 9.304 |

Therefore, it is confirmed that the composition of the present invention can be effectively applied to the treatment of eye diseases by significantly increasing the permeability to the vitreous body, retina and/or choroid upon eye drop administration, thereby maintaining a high drug concentration in the eye. In particular, unlike the conventional agents for treating macular degeneration that are injected into the vitreous body repetitively, the composition of the present invention can be conveniently administered as an eye drop formulation, thereby improving patients' compliance. In addition, since the composition of the present invention shows and maintains a rapid therapeutic and/or ameliorating effect from the beginning of administration, it can be usefully applied for the prevention, treatment, or improvement of macular degeneration.

### 2-4. Statistical Analysis

Normality was assumed for the experimental results, which confirmed with one-way ANOVA. If the ANOVA results were significant, a post-hoc test was performed using Dunnett's multiple comparison test. Statistical analysis was performed using Prism 7.04 (GraphPad Software Inc., San Diego, CA, USA). When the p value was less than 0.05, it was considered as being statistically significant.

1. A method for treating macular degeneration in a mammal, which comprises administering a therapeutically effective amount of enavogliflozin or a pharmaceutically acceptable salt thereof to the mammal in need thereof.

2. The method as claimed in claim 1, wherein enavogliflozin or a pharmaceutically acceptable salt thereof is delivered to a vitreous body, a retina, and/or a choroid when administered to the mammal in need thereof.

3. The method as claimed in claim 1, wherein the administering is carried out by administering an eye drop formulation comprising a therapeutically effective amount of enavogliflozin or a pharmaceutically acceptable salt thereof to the mammal in need thereof.

4. The method as claimed in claim 1, wherein the eye drop formulation further comprises one or more excipients selected from the group consisting of polyoxyl castor oil, polysorbate, D-α-tocopheryl polyethylene glycol succinate, and glycerin.

5. A pharmaceutical composition comprising a therapeutically effective amount of enavogliflozin or a pharmaceutically acceptable salt thereof and a pharmaceutically acceptable excipient, wherein the pharmaceutical composition is for treating macular degeneration.

6. The pharmaceutical composition as claimed in claim 5, wherein enavogliflozin or a pharmaceutically acceptable salt thereof is delivered to a vitreous body, a retina, and/or a choroid when the pharmaceutical composition is administered to the mammal in need thereof.

7. The pharmaceutical composition as claimed in claim 5, which is in the form of eye drop formulation.

8. The pharmaceutical composition as claimed in claim 5, wherein the pharmaceutically acceptable excipient is one or more selected from the group consisting of polyoxyl castor oil, polysorbate, D-α-tocopheryl polyethylene glycol succinate, and glycerin.
